(19) United States
(12) Patent Application Publication
(10) Pub. No.: US 2007/0202090 A1
(43) Pub. Date: Aug. 30, 2007

(54) Water Soluble Form Of Coenzyme Q10 In The Form Of An Inclusion Complex With Beta-Cyclodextrin, Process Of Preparing, And Use Thereof

(76) Inventors: Mirko Prosek, Ljubljana (SI); Andrej Smidovnik, Ljubljana (SI); Maja Fir, Smarje Pri Jelsah (SI); Monika Strazisar, Mojstrana (SI); Alenka Golc Wondra, Ljubljana (SI); Samo Andrensek, Ljubljana (SI); Janko Zmitek, Ljubljana (SI)

Correspondence Address: SMITH MOORE LLP, P.O. BOX 21927, GREENSBORO, NC, 27420, US

(21) Appl. No.: 11/569,110

(22) PCT Filed: May 10, 2005
(86) PCT No.: PCT/SI05/00013

§ 371 Date: Nov. 14, 2006

(30) Foreign Application Priority Data
May 18, 2004 (SI) P-200400144
Apr. 26, 2005 (SI) P-200500127

(51) Int. Cl. A61K 38/43; A61K 31/724; C08B 37/16

(52) U.S. Cl. 424/94.1; 514/58; 536/103

(57) ABSTRACT

An inclusion complex comprising coenzyme Q10 and β-cyclodextrin, its manner of preparation, and its use in products, such as, for example, pharmaceuticals, cosmetics, and aliments is disclosed. β-cyclodextrin is dissolved in water at a temperature elevated above room temperature, such as, for example, between 55° C. and water's boiling temperature. While stirring the β-cyclodextrin-water solution, coenzyme Q10 is added either in a solid form or a solution. Stirring is continued at the evaluated temperature and, then, either at room temperature or a temperature lower than room temperature. The complex enhances the water solubility of the coenzyme Q10 significantly. Also, the complex improves the bioavailability and prophylactic or therapeutical effectiveness of coenzyme Q10. Thus, the inclusion complex solves the problem of poor water-solubility of coenzyme Q10 that renders difficult a manufacture of effective preparations with this enzyme on a non-lipophilic base, as well as its addition to various alimentary, cosmetic, and/or pharmaceutical products.

This invention relates to a new water-soluble form of coenzyme Q10, to a process of preparing, and to the use thereof. Coenzyme Q10 of the present invention is in the form of an inclusion complex with β-cyclodextrin.

The invention relates to an inclusion complex of coenzyme Q10 with β-cyclodextrin. The invention, further relates to processes of preparing such a complex, and to the use of the latter in the form of separate preparations or as additives to pharmaceutical, cosmetic and alimentary products.

In the continuation of this text the following abbreviations (synonymes) shall be used:
for coenzyme Q10: CoQ10;
for the inclusion complex of coenzyme Q10 with β-cyclodextrin: CDQ10.

Coenzyme Q10 (CoQ10) is a lipophilic, water-insoluble substance, that is indispensable for the functioning of the human organism, since it is involved as coenzyme in numerous metabolic processes. Man ensures sufficient amounts of CoQ10 in the organism by means of synthesis and from the food. The intake of exogenous CoQ10 and other coenzymes Q (CoQ) into the human organism also influences the synthesis of endogenous CoQ10. The intake of sufficient amounts of exogenous CoQ10 and other coenzymes Q is critical, especially in the elderly, when the synthesis of endogenous CoQ10 becomes deficient, and the loss requires replenishment, by diet or by supplements in the form of various preparations. Often various food processing methods significantly deplete their CoQ10 content. Thus, the replenishment of the loss by means of supplementation is of the utmost importance. The addition of CoQ, especially CoQ10 into several essential foodstuffs by CoQ, especially CoQ10, is also of significant importance. The lipophilic character and the insolubility of CoQ, especially CoQ10, in water render difficult the manufacture of suitable formulations. For this reason there is a continuous need for the obtaining of a water-soluble form of ubiquinone with improved bioavailability, which would enable the manufacture of preparations possessing improved biopharmaceutical and nutritional CoQ10 properties. Such form should enable also the addition of CoQ10 into foodstuffs and other products.

Commercially available preparations contain CoQ10 either in the form of oily suspensions, for example in soft gelatine capsules, or in powdery formulations with various excipients, for example in hard capsules.

Owing to its lipophilic properties, CoQ10 is partially solubilized in oil-based preparations. Accordingly, it is more rapidly absorbed after food consumption, and the attained blood levels are relatively high. Several authors have, however, established that it is subsequently rapidly excreted from the organism via urine. Physiological effects are accordingly poorer than expected.

On the other hand, it is believed that in aqueous media dissolved CoQ10 is rapidly absorbed into the cells, for example muscle cells. Hence it is much slower excreted from the organism, and accordingly effective for a longer period. CoQ10 is in water substantially insoluble, so its bioavailability is poor. The effective use of CoQ10 as food supplement or in separate formulations is in need of a form enabling an enhanced water-solubility, and as such improved bioavailability and effectiveness. Investigations have shown a direct connection between the dissolution rate and the bioavailability of CoQ10. Water-solubility is particularly important for topical administration, for example into the oral cavity, on the skin and muscles, and the like.

For this reason, several authors have attempted in various ways to improve the water-solubility of CoQ10 in order to increase its bioavailability and effectiveness.

BioTakenaka Pharm. from Japan developed a special technique to improve the solubility of CoQ10, known as Bio-Coenzyme Q10 purum. The technique is based on a combination with the enzyme and enables a good solubility and a substantially increased bioavailability. Preparations on this base are marketed by Kampoyaki Research SDN BHD (Malaysia) in the form of gels, tablets or capsules. The combination with the enzyme renders this technique relatively exacting, expensive and rather sensitive.

The article A. Lutka, J. Pawlaczyk, Acta Poloniae Pharm.—Drug Res. 1995, 52, pp 379-386 discloses attempts to improve the water-solubility of CoQ10 by means of inclusion into various cyclodextrins. It has been known from the article J. Szejtli, J. Materials Chem. 1997, 7, pp 575-587, that the preparation of complexes of lipophilic substances with cyclodextrins increases their water-solubility. The authors tried to prepare inclusion complexes of CoQ10 with α-cyclodextrin, β-cyclodextrin, methyl β-cyclodextrin (0.97 and 1.8), hydroxypropyl cyclodextrin, and γ-cyclodextrin. It appears that they succeeded in the preparation of the complex of CoQ10 with y-cyclodextrin, possibly with substituted β-cyclodextrins also. No complex with a-cyclodextrin was obtained. Neither were they successful in the obtaining of a complex with β-cyclodextrin, which would be commercially most suitable owing to its low price, its characteristics, and the investigated hazards for humans. Except in the USA, β-cyclodextrin per se has been used in alimentation for a long time. It has been used in Japan without restrictions from 1983. Its use is approved in several European countries as well. Recently Food and Agriculture Organization and World Health Organization (WHO) jointly recommended 5 mg β-cyclodextrin per kg of body weight as maximum daily dose for human aministration—Article Z. H. Qi, C. T. Sikorski, Pharm. Technol. Europe 1002, 13 (11), pp 17-27. For all the utilization of relatively exacting equipment, the authors did not succeed in obtaining a water-soluble form of CoQ10 with the inexpensive and physiologically acceptable β-cyclodextrin.

The hitherto not appropriately solved problem is a suitable water-soluble form of coenzymes Q, especially CoQ10. A further problem is its preparation in an easy and economical manner. In spite of numerous investigations and results relating to the use of CoQ10, its utilization on a non-lipophilic base has therefore not been known as yet as an additive of food products, and cosmetic and pharmaceutical preparations. Neither have been known CoQ10 preparations, suitable to be added into such products, in a manner ensuring an appropriate concentrations of CoQ10.

The object and the aim of this invention are a water-soluble form of coenzyme Q10 with β-cyclodextrin, a process of preparation thereof, and its use in the form of separate preparations or as additive to pharmaceutical, cosmetic and alimentary products.

According to the present invention said object is achieved by a water-soluble inclusion complex of CoQ10 with β-cyclodextrin (CDQ10), a process of its preparation, and its use, as claimed in the independent claims.

### THE DRAWINGS REPRESENT:

FIG. 1: IR spectra of CDQ10, and of a physical mixture of β-cyclodextrin and CoQ10;

FIG. 2: DSC curve of β-cyclodextrin;

FIG. 3: DSC curve of CoQ10;

FIG. 4: DSC curves of a physical mixture of β-cyclodextrin and CoQ10, as well as of CDQ10;

FIG. 5: Powder X-ray diffractograms of CDQ10, and of a physical mixture of β-cyclodextrin and CoQ10.

The complexes are prepared in a novel and unobvious manner, by selecting conditions based on the characteristics of β-cyclodextrin and CoQ10, that render feasible the formation of the CDQ10 inclusion complex. The essential feature of the process of preparing CDQ10 is the dissolving of β-cyclodextrin in water at increased temperature, namely at a temperature exceeding room temperature, preferably at a temperature between 55° C. and the boiling temperature. Then follows, under vigorous stirring, the addition of CoQ10 in solid form or dissolved in a minimal quantity of a suitable, water-miscible, physiologically acceptable solvent in which CoQ10 is soluble, such as ethanol, acetone and the like. The inclusion complex is formed by inclusion of the CoQ10 molecule or its moieties into one or more β-cyclodextrin molecules. Owing to the structure, especially the length of the CoQ10 molecule, the latter is included into 1 to 10, or more β-cyclodextrin molecules. Accordingly, we use the ratio of CoQ10 to β-cyclodextrin to control the proportion and degree of CoQ10 complexation. A partial improvement of CoQ10 solubility and its bioavailabiliy is achieved even with sub-equimolar quantities of β-cyclodextrin with respect to CoQ10 (for example in the ratio 1:10). The proportion of the complexated CoQ10, and the complexation degree (the ratio of CoQ10 to β-cyclodextrin in the inclusion complex) increase with the augmentation of said ratio in favour of β-cyclodextrin, which virtually has no upper limit. For this reason we use an optional ratio of β-cyclodextrin to CoQ10, namely up to several tenfold, preferaby up to thirtyfold excesses of β-cyclodextrin, when we desire to use the complex with other substances, which tend to form complexes with β-cyclodextrin. Normally we use ratio of 1:10 to 10:1, preferably 1:5 to 5:1, more preferably about 1:1, which yield optimal results with regard to the properties of the inclusion complex and the synthesis costs. Preferably, CoQ10 is added in solid form, and a 10-20% molar excess of β-cyclodextrin to CoQ10 is used. Vigorous stirring is continued till the disappearance of CoQ10, and the beginning of precipitation of the inclusion complex CDQ10. The reaction mixture is cooled and CDQ10 is isolated by means of decantation, filtration or water evaporation. Optionally CDQ10 is dried. It may be used, however, in the form of dissolved or undried CDQ10 complexes, as the manufacture is performed in an aqueous medium. For this reason the obtained mixture does not contain noxious solvents. The yield of CDQ10 is practically quantitative. CoQ10 in the form of the obtained CDQ10 shows an improved solubility in aqueous media. It exceeds the solubility required for the daily dose of CoQ10 administered to an adult in the form of separate preparations, or in the form of additives to food and other products.

Hence this invention also relates to preparations containing the CDQ10 complex, as well as to its use as food additive or independently for alimentary, prophylactic, therapeutical, cosmetic and other purposes.

The improved properties of CoQ10 in the form of CDQ10 enable a technically simple and economically suitable production of formulations, which are not lipid-based. They show suitable dissolution and solubility values in aqueous media, thus enhancing the absorption in tissues and improving physiological effects. Furthermore, this makes them widely applicabile for various purposes, or for various administration routes, such as oral, buccal, and topical. Preparations may be solid, semisolid, or liquid, for example in the form of capsules, tablets, powders, syrups, solutions, gels, drops, creams, or in other formulations or delivery systems. They are utilized to achieve various systemic and local effects, inter alia in prophylaxis and therapy of cardiovascular diseases, hypercholesterolemia, diabetes, cancer, and immune disorders, muscular dystrophy, diseases of the oral cavity, especially the gingivae, and the like. An aqueous solution is easily prepared for gingival rinsing, and may be supplemented by further ingredients to enhance its acceptability to the user. CDQ10 may be added to toothpastes as well, and has a beneficial effect on the gingivae. The same applies for various creams and solutions for external use. The present invention relates further to the use of the CDQ10 complex as an ingredient of food products, admixed in an isolated or unisolated form into the food or into alimentary products, such as milk, yoghurts, cottage cheese, cheese, butter and other dairy products, marmalade and jams, fruit and vegetable juices and nectars, and various beverages, compotes or stewed fruit, groats, cereal products, chocolate products, teas, honey products, meat products, and the like. Inclusion complexes of this invention, especially as CDQ10, may be incorporated into said products during one of the processing steps in the manufacture of the product. In the case of liquid, semisolid, and semiliquid products also after the finishing of the product. The possibility to add for example CDQ10 at the end of the process represents a further advantage, as it enables a simple organization and targeting of the production to the basic product, or a product with added CDQ10, or both. Similarly, CDQ10 may be added to alimentary or other products on the administration site or immediately before use, by incorporation of the complex into said product, in a formulation ready for use, for example in the form of a solution, syrup, drops, powder, capsules, and the like.

Accordingly, a sufficient amount of CDQ10 may be added to milk or yoghurt, to ensure for an adult the intake of a daily dose, or even multiple daily doses of CoQ10 in one ration. Namely, the solubility of CoQ10 in the form of CDQ10, for example in milk at room temperature, is about 2500 times the solubility of CoQ10 alone. Thus, at room temperature are dissolved about 8.5 mg CoQ10 in the form of CDQ10 in 1 g of milk. In such a manner the CoQ10 content in milk or yoghurts may be increased even 5000 times or more, as natural milk contains about 1.7×10⁻³ mg CoQ10/g of milk. In several types of milk or dairy products the content is scarser, even under the detection limit by conventional methods. Hence the possibility to add CoQ10 is even more important. Yoghurt or milk suplemented by CDQ10 are prepared preferably in such a manner, that they are admixed under stirring with the desired CDQ10 amount, for example 30-150 mg CoQ10, in the form of a complex in a suitable formulation. Preferably the complex is added in undried form or as a reaction mixture, preferably in the form of a reaction mixture in an appropriate formulation, such as a solution, syrup, drops, and the like. The milk and yoghurt with added CDQ10 may be ingested, or conveniently packed if the addition is performed during manufacture.

It is equally possible, owing to the solubility of CDQ10, to add CDQ10 to orange, strawberry, peach and other juices, nectars and beverages in optional quantities. Preferred amounts are conventional daily doses up to 150 mg of CoQ10 in the form of CDQ10, by means of dissolving the CDQ10 complex under stirring at the usual utilization temperature, in the juice, nectar or the beverage, prior to the consumation or the packaging. For the addition, the complex is used in isolated, dried or undried forms, or unisolated. Preferably, the complex is used undried or unisolated.

In an analoguous manner CDQ10 is added to semisolid products. CDQ10 is therefore admixed prior to the use or packaging into a toothpaste, liver pate, honey, marmalade, jam, and the like, by means of admixing, under stirring, the desired amount of the isolated, dried or undried, or unisolated CDQ10 in a suitable form.

A further aspect of this invention is the use of the CDQ10 complex and products thereof, relating to the functions of the human organism linked to the coenzyme Q10.

The invention is illustrated but not limited by the working examples.

Used were: β-cyclodextrin from Roquette, France and CoQ10 from Vivati Pharma GmbH, Germany.

### EXAMPLE 1

### Preparation of the CDQ10 Complex

a) Preparation Procedure

β-Cyclodextrin (1.575 g, 1.39 mmole) was dissolved in 13 mL of distilled water at 70° C. The dissolved β-cyclodextrin was admixed with 1.00 g (1.16 mmole) of CoQ10, and the mixture was stirred for 20 minutes at 70° C. After stirring for approximately 1 minute the CDQ10 complex started to precipitate. The amount of the precipitate increased for about 20 minutes. The reaction mixture was kept stirring for about 10 hours at 60-70° C., and several hours at room temperature, till the formation of a homogenous paste. Then the obtained mixture was centrifugated, the phases were separated, the suspension was washed with a small amount of water and dried at 30-35° C. under reduced pressure. The yield was 1.9 g of the CDQ10 complex in the form of a yellow-orange powder, decomposing at a temperature over 273° C. The product was identified by means of IR spectroscopy, thermal analysis (DSC), and powder X-ray analysis.

b) IR Spectroscopy

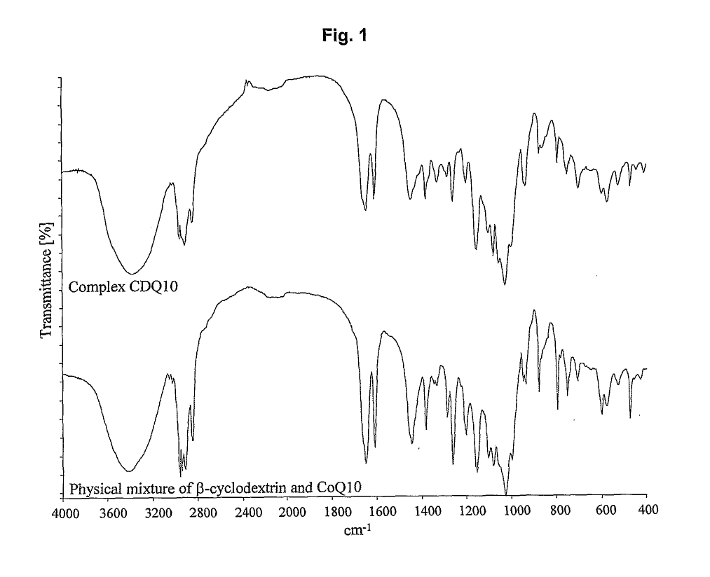

The comparison of the IR spectra on FIG. 1 and Table 1, representing the wave numbers for the bands, which are different in the two spectra, shows the differences between the physical mixture of β-cyclodextrin with CoQ10, and the inclusion complex CDQ10. The spectra were obtained on a FT-IR spectrometer SPECTRUM 1000 (Perkin-Elmer).

TABLE 1
Comparison of wave numbers for selected bands in the IR spectrum
of CDQ10 and the physical mixture of β-cyclodextrin and CoQ10,
and the difference between the individual bands.

| Bands for CDQ10 | Bands for the physical | |
|---|---|---|
| [cm⁻¹] | mixture [cm⁻¹] | Shift [cm⁻¹] |
| 3390.46 | 3418.53 | 28.07 |
| / | 2944.11 | |
| 2916.44 | 2913.28 | 2.16 |
| 1448.43 | 1446.53 | 1.9 |
| / | 1348.25 | |
| 1334.00 | 1336.56 | 2.56 |
| 1289.78 | 1287.60 | 2.18 |
| 1230.76 | 1227.97 | 2.79 |
| 1057.34 | / | |
| 1028.35 | 1026.07 | 2.28 |
| 1001.39 | 995.80 | 5.59 |
| 942.65 | 945.45 | 2.80 |
| 861.53 | / | |
| / | 780.41 | |
| 703.52 | 707.40 | 3.88 |
| 598.60 | 601.37 | 2.77 |
| 575.05 | 579.42 | 4.37 |
| 444.75 | / | |
| / | 425.17 | |
| 411.33 | / | |

c) Thermal Analysis (DSC)

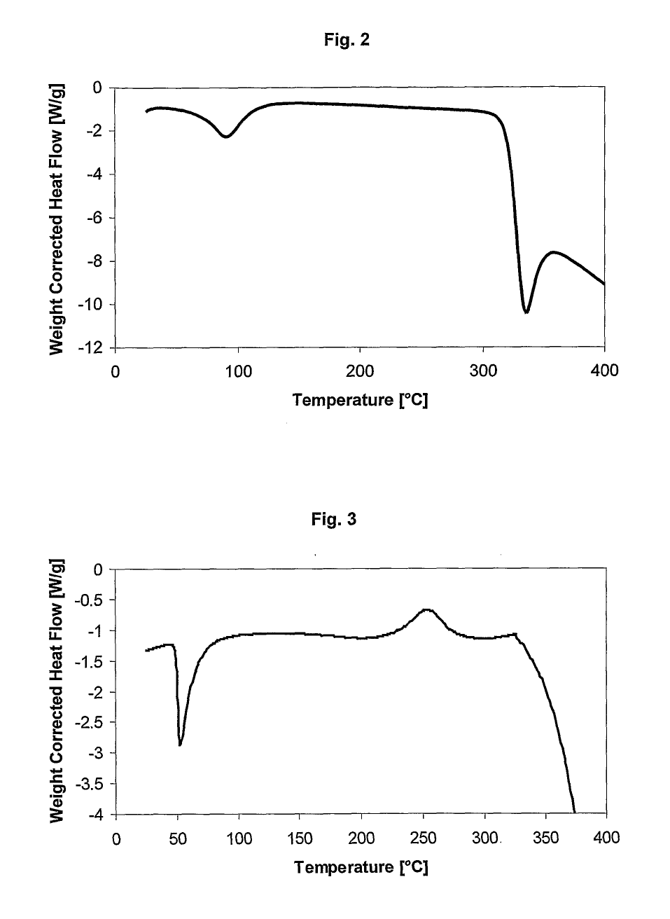

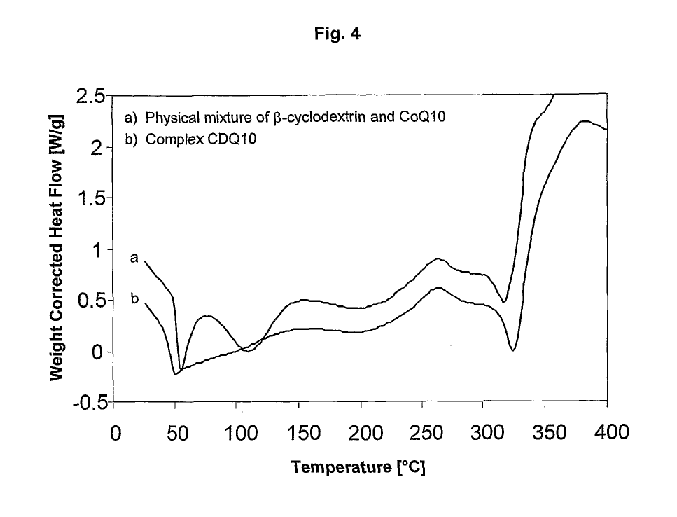

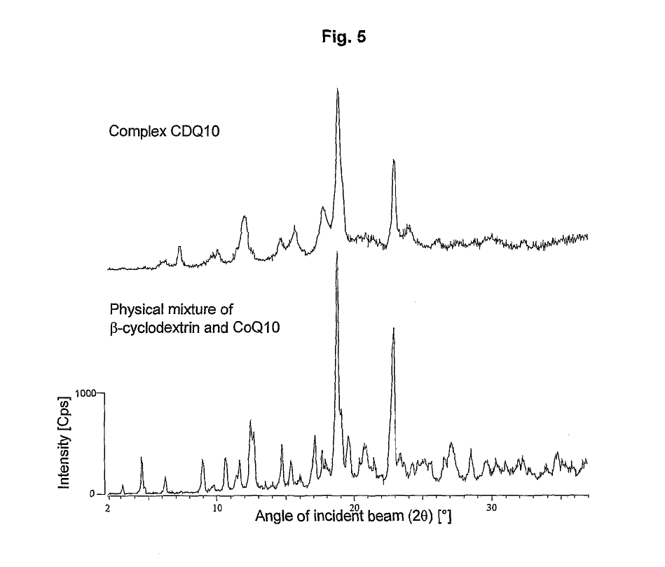

FIGS. 2, 3 and 4 show DSC curves of: β-cyclodextrin, CoQ10, their physical mixtures, and of the CDQ10 complex. The curves were obtained in a thermal analysis system SDT 2960 (TA Instruments Inc.). Characteristic are the differences in the peaks' shifts and the curves' shapes, especially at about 50 and 330° C. and between 100 and 150° C.

d) Powder X-ray Analysis

The differences between the diffractograms of the CDQ10 complex and the physical mixture of CoQ10 and β-cyclodextrin on FIG. 5, and the differences between the angles, mesh distances and peaks' intensities of CoQ10 and β-cyclodextrin, their equimolar physical mixture, and of CDQ10, as represented in Tables 2-5, confirm the formation of the CDQ10 inclusion complex.

TABLE 2
Angles, distances and intensities in the powder X-ray diffractogram
of β-cyclodextrin (A. of inc. b. = Angle of incident beam).

| A. of inc. b. | D | Intensity | Intensity % |
|---|---|---|---|
| 2θ [°] | [Å] | [Cps] | [%] |
| 4.478 | 19.717 | 1518 | 37.2 |
| 6.232 | 14.170 | 414 | 10.1 |
| 8.993 | 9.826 | 1528 | 37.4 |
| 9.731 | 9.081 | 92 | 2.3 |
| 10.664 | 8.289 | 447 | 11.0 |
| 11.622 | 7.608 | 488 | 12.0 |
| 12.480 | 7.087 | 918 | 22.5 |
| 12.682 | 6.974 | 1098 | 26.9 |
| 13.540 | 6.534 | 285 | 7.0 |
| 14.756 | 5.998 | 967 | 23.7 |
| 15.376 | 5.758 | 420 | 10.3 |
| 16.072 | 5.510 | 258 | 6.3 |
| 16.909 | 5.239 | 450 | 11.0 |
| 17.111 | 5.178 | 790 | 19.4 |
| 17.675 | 5.014 | 1115 | 27.3 |
| 17.960 | 4.935 | 651 | 15.9 |
| 18.830 | 4.709 | 4082 | 100.0 |
| 19.572 | 4.532 | 716 | 17.5 |
| 20.729 | 4.281 | 879 | 21.5 |
| 20.911 | 4.245 | 800 | 19.6 |
| 21.227 | 4.182 | 612 | 15.0 |
| 21.520 | 4.126 | 347 | 8.5 |
| 22.064 | 4.025 | 177 | 4.3 |
| 22.716 | 3.911 | 1076 | 26.4 |
| 23.440 | 3.792 | 364 | 8.9 |
| 23.723 | 3.747 | 318 | 7.8 |
| 24.299 | 3.660 | 446 | 10.9 |
| 25.048 | 3.552 | 539 | 13.2 |
| 25.280 | 3.520 | 493 | 12.1 |
| 25.679 | 3.466 | 491 | 12.0 |
| 26.657 | 3.341 | 679 | 16.6 |
| 27.064 | 3.292 | 869 | 21.3 |
| 27.600 | 3.229 | 318 | 7.8 |
| 28.576 | 3.121 | 336 | 8.2 |
| 29.500 | 3.025 | 336 | 8.2 |
| 30.222 | 2.955 | 365 | 8.9 |
| 30.848 | 2.896 | 320 | 7.8 |
| 31.047 | 2.878 | 389 | 9.5 |
| 32.026 | 2.792 | 773 | 18.9 |
| 32.858 | 2.723 | 352 | 8.6 |
| 34.066 | 2.630 | 382 | 9.4 |
| 34.560 | 2.593 | 560 | 13.7 |
| 34.833 | 2.573 | 845 | 20.7 |
| 35.329 | 2.538 | 478 | 11.7 |
| 35.801 | 2.506 | 383 | 9.4 |
| 36.637 | 2.451 | 457 | 11.2 |

TABLE 3
Angles, distances and intensities in the powder X-ray diffractogram
of CoQ10 (A. of inc. b. = Angle of incident beam).

| A. of inc. b. | D | Intensity | Intensity % |
|---|---|---|---|
| 2θ [°] | [Å] | [Cps] | [%] |
| 3.123 | 28.269 | 426 | 8.7 |
| 4.655 | 18.967 | 366 | 7.5 |
| 6.204 | 14.234 | 174 | 3.6 |
| 7.735 | 11.420 | 35 | 0.7 |
| 9.152 | 9.654 | 58 | 1.2 |
| 10.893 | 8.116 | 130 | 2.7 |
| 11.361 | 7.782 | 416 | 8.5 |
| 11.729 | 7.539 | 224 | 4.6 |
| 12.210 | 7.243 | 129 | 2.6 |
| 12.444 | 7.107 | 166 | 3.4 |
| 12.902 | 6.856 | 77 | 1.6 |
| 13.840 | 6.393 | 152 | 3.1 |
| 14.003 | 6.319 | 213 | 4.4 |
| 14.431 | 6.133 | 173 | 3.6 |
| 14.725 | 6.011 | 241 | 4.9 |
| 15.599 | 5.676 | 262 | 5.4 |
| 17.144 | 5.168 | 334 | 6.9 |
| 18.176 | 4.877 | 280 | 5.7 |
| 18.698 | 4.742 | 4872 | 100.0 |
| 19.046 | 4.656 | 1890 | 38.8 |
| 20.376 | 4.355 | 708 | 14.5 |
| 21.027 | 4.221 | 237 | 4.9 |
| 21.824 | 4.069 | 329 | 6.8 |
| 22.868 | 3.886 | 3600 | 73.9 |
| 23.369 | 3.803 | 651 | 13.4 |
| 24.652 | 3.608 | 578 | 11.9 |
| 25.714 | 3.462 | 253 | 5.2 |
| 26.949 | 3.306 | 243 | 5.0 |
| 27.322 | 3.261 | 633 | 13.0 |
| 28.584 | 3.120 | 602 | 12.4 |
| 29.694 | 3.006 | 593 | 12.2 |
| 30.401 | 2.938 | 555 | 11.4 |
| 30.849 | 2.896 | 327 | 6.7 |
| 31.353 | 2.851 | 237 | 4.9 |
| 32.286 | 2.770 | 462 | 9.5 |
| 32.756 | 2.732 | 235 | 4.8 |
| 33.080 | 2.706 | 304 | 6.2 |
| 33.680 | 2.659 | 193 | 4.0 |
| 34.511 | 2.597 | 192 | 3.9 |
| 35.844 | 2.503 | 189 | 3.9 |

TABLE 4
Angles, distances and intensities in the powder X-ray diffractogram
of a physical mixture of β-cyclodextrin and CoQ10 (A.
of inc. b. = Angle of incident beam).

| A. of inc. b. | D | Intensity | Intensity % |
|---|---|---|---|
| 2θ [°] | [Å] | [Cps] | [%] |
| 3.045 | 28.992 | 83 | 3.5 |
| 4.463 | 19.782 | 351 | 14.7 |
| 6.167 | 14.321 | 159 | 6.7 |
| 8.928 | 9.896 | 331 | 13.9 |
| 9.712 | 9.100 | 92 | 3.9 |
| 10.624 | 8.320 | 360 | 15.1 |
| 11.382 | 7.768 | 186 | 7.8 |
| 11.608 | 7.617 | 337 | 14.2 |
| 12.430 | 7.115 | 728 | 30.6 |
| 13.510 | 6.549 | 122 | 5.1 |
| 13.973 | 6.333 | 96 | 4.0 |
| 14.671 | 6.033 | 489 | 20.5 |
| 15.324 | 5.777 | 318 | 13.4 |
| 16.013 | 5.530 | 177 | 7.4 |
| 17.074 | 5.189 | 576 | 24.2 |
| 17.629 | 5.027 | 432 | 18.2 |
| 17.840 | 4.968 | 343 | 14.4 |
| 18.718 | 4.737 | 2380 | 100.0 |
| 19.080 | 4.648 | 782 | 32.9 |
| 19.550 | 4.537 | 572 | 24.0 |
| 20.365 | 4.357 | 329 | 13.8 |
| 20.756 | 4.276 | 488 | 20.5 |
| 21.463 | 4.137 | 341 | 14.3 |
| 21.956 | 4.045 | 191 | 8.0 |
| 22.860 | 3.887 | 1635 | 68.7 |
| 23.382 | 3.801 | 402 | 16.9 |
| 23.641 | 3.760 | 310 | 13.0 |
| 24.254 | 3.667 | 302 | 12.7 |
| 24.666 | 3.606 | 324 | 13.6 |
| 25.210 | 3.530 | 349 | 14.7 |
| 25.564 | 3.482 | 304 | 12.8 |
| 26.620 | 3.346 | 355 | 14.9 |
| 27.055 | 3.293 | 507 | 21.3 |
| 28.535 | 3.126 | 441 | 18.5 |
| 29.608 | 3.015 | 320 | 13.4 |
| 30.299 | 2.947 | 331 | 13.9 |
| 31.000 | 2.882 | 318 | 13.4 |
| 31.942 | 2.799 | 342 | 14.4 |
| 32.235 | 2.775 | 358 | 15.0 |
| 32.726 | 2.734 | 256 | 10.8 |
| 33.040 | 2.709 | 204 | 8.6 |
| 33.925 | 2.640 | 276 | 11.6 |
| 34.748 | 2.580 | 408 | 17.1 |
| 35.087 | 2.555 | 337 | 14.2 |
| 35.905 | 2.499 | 261 | 11.0 |

TABLE 5
Angles, distances and intensities in the powder X-ray diffractogram
of the CDQ10 inclusion complex of CoQ10 with β-cyclodextrin
(A. of inc. b. = Angle of incident beam).

| A. of inc. b. | D | Intensity | Intensity % |
|---|---|---|---|
| 2θ [°] | [Å] | [Cps] | [%] |
| 2.88 | 30.652 | 24 | 1.3 |
| 4.313 | 20.470 | 22 | 1.2 |
| 4.702 | 18.779 | 41 | 2.3 |
| 5.076 | 17.395 | 28 | 1.6 |
| 5.68 | 15.546 | 57 | 3.2 |
| 5.917 | 14.925 | 89 | 5.0 |
| 6.241 | 14.151 | 98 | 5.5 |
| 7.261 | 12.165 | 241 | 13.5 |
| 9.040 | 9.774 | 87 | 4.9 |
| 9.720 | 9.092 | 165 | 9.3 |
| 10.020 | 8.820 | 201 | 11.3 |
| 11.440 | 7.729 | 222 | 12.5 |
| 12.000 | 7.369 | 532 | 29.9 |
| 12.525 | 7.061 | 212 | 11.9 |
| 14.570 | 6.074 | 314 | 17.6 |
| 15.610 | 5.672 | 427 | 24.0 |
| 17.688 | 5.010 | 625 | 35.1 |
| 18.773 | 4.723 | 1781 | 100.0 |
| 20.798 | 4.267 | 361 | 20.3 |
| 21.325 | 4.163 | 330 | 18.5 |
| 22.892 | 3.882 | 1089 | 61.1 |
| 23.932 | 3.715 | 418 | 23.5 |
| 26.192 | 3.399 | 311 | 17.5 |
| 28.784 | 3.099 | 296 | 16.6 |
| 29.520 | 3.023 | 319 | 17.9 |
| 32.375 | 2.763 | 299 | 16.8 |
| 33.118 | 2.703 | 273 | 15.3 |
| 35.120 | 2.553 | 326 | 18.3 |
| 35.280 | 2.542 | 313 | 17.6 |
| 36.821 | 2.439 | 335 | 18.8 |

### EXAMPLE 2

### Comparison of Milk-solubility of CoQ10 and its CDQ10

β-Cyclodextrin (1.575 g, 1.39 mmole) was dissolved in 13 mL of distilled water at 60-70° C. Then 1.00 g (1.16 mmole) of CoQ10 were added to the dissolved β-cyclodextrin, and the mixture was stirred for 20 minutes at 60-70° C. After stirring for some minutes the CDQ10 complex started to precipitate. The reaction mixture was kept stirring for about 8 hours at 60-70° C., and about 2 hours at room temperature, till the formation of a homogenous paste. Then the obtained mixture was under stirring at room temperature added to fresh milk containing 3.5% of milk fat (Ljubljanske mlekarne) till saturation. Into another milk aliquot was added under identical conditions CoQ10 till saturation. The CoQ10 contents of the two samples were determined by the filtration of the samples, followed by the extraction of 10 g of each filtrate 7 times with 2 mL of n-hexane and once with 2 mL of chloroform, the evaporation under reduced pressure of the solvents from the combined organic fractions, the dissolution of the oily residues in ethanol (3 mL), and the dilution to 1:1000 (vol/vol), and HPLC analysis (Thermo Separation Products GmbH—Surveyor gradient pump, automatic sampler with a 20 μL loop, UV/VIS detector, HyperClone ODS column (Phenomenex), 150×4.6 mm, particle size 5 μm; mobile phase MeOH: EtOH: 2-propanol in a ratio of 70:15:15 (vol/vol/vol), room temperature 25 ° C., flow rate 1 mL/minute; software OS2). The retention time of CoQ10 was 10.7±0.5 minutes. The results are represented in Table 6.

TABLE 6
The CoQ10 content in milk: a) without additives, b)
after the addition of CoQ10 till saturation, c) after
the addition of the CDQ10 complex till saturation.

Sample

| | a | b | c |
|---|---|---|---|
| Concentration of CoQ10 in ug/g | 1.7 | 3.2 | 8.5.10³ |

### EXAMPLE 3

### Preparation of Dairy Products and Fruit Juices with the Addition of CDQ10

β-Cyclodextrin (7.90 g, 6.95 mmole) was dissolved in distilled water (68 mL) at 65-70° C., and under stirring was added CoQ10 (5.00 g, 5.79 mmole). After about 30 minutes the CDQ10 complex precipitated. The reaction mixture was kept stirring for about 10 hours at 70° C., and 10 hours at room temperature, till the formation of an intensive yellow coloured paste. Then equal aliquots (each 500 μL containing about 37 mg of CoQ10 in the form of CDQ10) of the obtained mixture were admixed to about 200 g each: milk (3.5% of milk fat, Ljubljanske miekarne), liquid yoghurt (1.3% of milk fat, Ljubljanske miekarne), fruit yoghurt (peach, Ljubljanske miekarne) and orange juice (100%, Rauch). The obtained preparations were organoleptically compared with non-lemented supplemented milk, liquid yoghurt, fruit yoghurt and orange juice, as well as with those supplemented each with about 35-37 mg CoQ10. As CoQ10 was not dissolved in said products, they were not included in the tests. The tests were performed by 10-21 randomly chosen consumers. The results are represented in Table 7 and show the good acceptability of the products supplemented with CDQ10.

TABLE 7
The results of organoleptic testing of products supplemented
or not supplemented with CoQ10 in the form of CDQ10. Represented
are the higher acceptability portions for the individual preparations
- more acceptable a) sample without additive, b) sample with
added CDQ10, c) no difference was established between the sample
without additive or with the added CDQ10.(Appear. = Appearance,
m.f. = milk fat)

| Product | Acceptability | Appear. | Smell | Taste |
|---|---|---|---|---|
| Milk | a | 57.1 | 33.3 | 33.3 |
| (3.5% m.f.) | b | 14.3 | 23.8 | 33.3 |
| | c | 28.6 | 42.9 | 33.3 |
| Liquid | a | 63.6 | 36.4 | 36.4 |
| Yoghurt | b | 27.3 | 27.3 | 45.5 |
| (1.3% m.f.) | c | 9.1 | 36.4 | 18.2 |
| Orange | a | 72.7 | 27.3 | 36.4 |
| juice | b | 9.1 | 45.5 | 45.5 |
| (100%) | c | 18.2 | 27.3 | 18.2 |
| Fruit | a | 10.0 | 20.0 | 10.0 |
| yoghurt | b | 20.0 | 20.0 | 20.0 |
| (peach) | c | 70.0 | 60.0 | 70.0 |

### EXAMPLE 4

### Preparation of Toothpaste with the Addition of CDQ10

β-Cyclodextrin (7.90 g, 6.95 mmole) was dissolved in distilled water (68 mL) at 60-70° C. Then CoQ10 (5.00 g, 5.79 mmole) was added to the dissolved β-cyclodextrin, and the mixture was stirred for about 8 hours at 60-70° C. and about 2 hours at room temperature, till the formation of a homogenous, spreadable mixture. A portion of the obtained mixture (8.1 g) was at room temperature homogenously admixed to a toothpaste (49 g, Biodent, Lek). The latter contained about 1% CoQ10 in the form of the CDQ10 inclusion complex, and was used like a customary toothpaste.

### EXAMPLE 5

### Preparation of CDQ10 in a Ratio 1:5

β-Cyclodextrin (1.501 g, 1.32 mmole) was dissolved in 15 mL of distilled water at 70° C. Then 228.4 mg (0.265 mmole) of CoQ10 were added to the dissolved β-cyclodextrin, and the mixture was vigorously stirred for 20 minutes at 70° C. After stirring for approximately 1 minute, the CDQ10 complex started to precipitate, and the amount of the precipitate increased for about 20 minutes. The reaction mixture was kept stirring for about 10 hours at 60-70° C. and for 2 hours at room temperature till the formation of a homogenous paste. The obtained mixture was collected by filtration, the precipitate was washed with a small amount of water and dried at 25-30° C. under reduced pressure. The yield was 1.1 g of CDQ10 in the form of a yellow-orange powder, m.p. 295-297° C. The product was identified by means of IR spectroscopy, thermal analysis (DSC), and powder X-ray analysis.

The novel water-soluble coenzyme Q10 form according to this invention is therefore an inclusion complex of β-cyclodextrin with coenzyme Q10 in an optional ratio, preferably in a molar ratio of β-cyclodextrin to coenzyme Q10 within the range of (several 10):1, preferably up to 30:1, preferably within the range of 1:10 to 10:1, more preferably 1:5 to 5:1, especially preferably within the ratio about 1:1. The β-cyclodextrin/coenzyme Q10 inclusion complexes are isolated or unisolated from the reaction mixture.

The process of preparation of the novel water-soluble form is characterised in that β-cyclodextrin is dissolved in water at increased temperature, between 30° C. and the boiling temperature, preferably between 55° C. and the boiling temperature, then coenzyme Q10 is added either in solid form or dissolved in an appropriate solvent. At first the stirring is continued at increased temperature, then at room temperature or lower than room temperature. The molar ratio of β-cyclodextrin to coenzyme Q10 is within the range of (several 10):1, preferably up to 30:1, preferably within the range of 1:10 to 10:1, more preferably 1:5 to 5:1, especially preferably within the ratio about 1:1.

The novel water-soluble coenzyme Q10 form according to this invention may be used as additive to pharmaceutical, cosmetic and alimentary products. It may be added to said products either isolated or unisolated from the reaction mixture, any time during the process of their manufacture. It is, however, preferably added to the manufactured product.

1. A new water-soluble coenzyme Q10 form comprising an inclusion complex comprising β-cyclodextrin and coenzyme Q10, wherein the β-cyclodextrin:coenzyme Q10 molar ratio comprises between about 1:10 and about 10:1.

2. The new water-soluble coenzyme Q10 form according to claim 1, wherein the inclusion complex comprises either an isolate or a component of a reaction mixture.

3. The new water-soluble coenzyme Q10 form according to claim 1, wherein the solubility of coenzyme Q10 in an aqueous media is improved in the form of the inclusion complex.

4. A process for preparing a new water-soluble coenzyme Q10 form comprising an inclusion complex comprising β-cyclodextrin and coenzyme Q10 at a β-cyclodextrin:coenzyme Q10 molar ratio comprising between about 1:10 and about 10:1, the process comprising the steps of:
dissolving β-cyclodextrin in water at an elevated temperature between about 30° C. and about water's boiling temperature;
adding coenzyme Q10 either in a solid form or as a solute dissolved in an appropriate solvent;
stirring at the elevated temperature; and
stirring at room temperature or a reduced temperature lower than room temperature.

5. The process according to claim 4, wherein the β-cyclodextrin:coenzyme Q10 molar ratio comprises between about 1:5 and about 5:1.

6. A use of new water-soluble coenzyme Q10 form comprising an inclusion complex comprising β-cyclodextrin and coenzyme Q10 at a β-cyclodextrin:coenzyme Q10 molar ratio comprising between about 1:10 and about 10:1 as an additive to any one of a pharmaceutical product, a cosmetic product, or an alimentary product.

7. The use of new water-soluble coenzyme Q10 form according to claim 6, wherein the new water-soluble coenzyme Q10 form is added to any one of the products, either as an isolate or a component of a reaction mixture.

8. The use of new water-soluble coenzyme Q10 form according to claim 6, wherein the new water-soluble coenzyme Q10 form is added to the product at any time during its manufacture or to the manufactured product.

9. A new water-soluble coenzyme Q10 form comprising an inclusion complex comprising β-cyclodextrin and coenzyme Q10, wherein the β-cyclodextrin:coenzyme Q10 molar ratio comprises up to about 30:1.

10. The new water-soluble coenzyme Q10 form according to claim 9, wherein the inclusion complex comprises either an isolate a component of a reaction mixture.

11. The new water-soluble coenzyme Q10 form according to claim 9, wherein the solubility of coenzyme Q10 in an aqueous media is improved in the form of the inclusion complex.

12. A process for preparing a new water-soluble coenzyme Q10 form comprising an inclusion complex comprising β-cyclodextrin and coenzyme Q10 at a β-cyclodextrin:coenzyme Q10 molar ratio comprising up to about 30:1, the process comprising the steps of:
dissolving β-cyclodextrin in water at an elevated temperature between about 30° C. and about water's boiling temperature;
adding coenzyme Q10 either in a solid form or as a solute dissolved in an appropriate solvent;
stirring at the elevated; and
stirring at room temperature or a reduced temperature lower than room temperature.

13. The process according to claim 12, wherein the β-cyclodextrin:coenzyme Q10 molar ratio comprises up to about 10:1.

14. A use of new water-soluble coenzyme Q10 form comprising an inclusion complex comprising β-cyclodextrin and coenzyme Q10 at a β-cyclodextrin:coenzyme Q10 molar ratio comprising up to about 30:1 as an additive to any one of a pharmaceutical product, a cosmetic product, or an alimentary product.

15. The use of new water-soluble coenzyme Q10 form according to claim 14, wherein the new water-soluble coenzyme Q10 form is added to any one of the products either as an isolate a component of a reaction mixture.

16. The use of new water-soluble coenzyme Q10 form according to claim 14, wherein the new water-soluble coenzyme Q10 form is added to the products at any time during its manufacture or to the manufactured product.

17. The new water-soluble coenzyme Q10 form according to claim 1, wherein the β-cyclodextrin: coenzyme Q10 molar ratio comprises between about 1:5 and about 5:1.

18. The new water-soluble coenzyme Q10 form according to claim 1, wherein the β-cyclodextrin:coenzyme Q10 molar ratio comprises about 1:1.

19. The process according to claim 4, wherein the elevated temperature comprises between about 55° C. and about water's boiling temperature.

20. The process according to claim 12, wherein the elevated temperature comprises between about 55° C. and about water's boiling temperature.
